(19) United States
(12) Patent Application Publication
(10) Pub. No.: US 2021/0193276 A1
(43) Pub. Date: Jun. 24, 2021

(54) INTEGRATED HEALTHCARE MONITORING SYSTEM AND METHOD THEREFOR

(71) Applicant: Yubi Health Inc., Shakopee, MN (US)

(72) Inventors: Rahul Kumar Goyal, Shakopee, MN (US); Vivek Goel, Shakopee, MN (US); Chris E. Stout, Shakopee, MN (US); Rahul Deep, Shakopee, MN (US)

(21) Appl. No.: 17/130,078

(22) Filed: Dec. 22, 2020

Related U.S. Application Data
(60) Provisional application No. 62/952,959, filed on Dec. 23, 2019.

(51) Int. Cl. G16H 10/60; G16H 50/30; G16H 10/20

(52) U.S. Cl. CPC G16H 10/60; G16H 10/20; G16H 50/30

(57) ABSTRACT

Disclosed is an integrated health care monitoring method and system. The system fetches the employer data that provides initial indication of potential employees that have behavioral health issues and requests a member to undergo one or more a wellness assessments to identify member archetypes. The system scores each question mapped to an archetype and then the overall scores across the archetypes are determined to derive the major/minor archetype based on employer data and wellness assessments. The system results from with behavioral health assessment to provide high likelihood of mental health issues with underlying issues based on archetypes. The archetypes can further trigger genetics or microbiome tests or biosensors or activity data to confirm the exact underlying. Based on the underlying issue, appropriate recommendations are suggested for the mental behavior wellness of the member.

### CROSS-REFERENCE TO RELATED APPLICATION

This application claims the benefit of and priority to U.S. Provisional Patent Application No. 62/952,959 entitled “AN INTEGRATED HEALTHCARE MONITORING SYSTEM AND METHOD THEREFOR,” filed on Dec. 23, 2019, the contents of which being incorporated by reference in their entirety herein.

### TECHNICAL FIELD

The present disclosure relates generally to health awareness systems and more specifically, methods and systems to identify users having mental health problems along with the underlying issues triggering the mental health problem.

### BACKGROUND

In recent years, people have started to focus more on their health, such as, by regularly monitoring to check for various health conditions. Consequently, such monitoring of health enables people to improve their health, by taking corrective measures such as by improving dietary intake, incorporating more physical activities into daily routine, following a medication regimen and so forth. Traditionally, such monitoring of the health is performed by undertaking pathological tests, for example, through blood tests, urine tests, electrocardiogram (or ECG), and the like. However, it will be appreciated that the health of an individual not only depends on a physical health thereof, but also various other factors such as mental health, social behavior, hereditary factors, temptations towards consumption of harmful substances (such as alcohol), and so forth. Consequently, conventional techniques that focus on pathological tests to monitor health of an individual are usually ineffective at monitoring a holistic health of the individual, such as, for predicting one or more diseases that the individual may likely develop in a near future.

Various prior art solutions have been contemplated to overcome such drawbacks associated with conventional techniques for monitoring health of individuals. In one prior art solution, provided is a detecting, monitoring and reporting system includes a sensor device which generates data indicative of physiological parameters of an individual and data derived therefrom when placed in proximity with the body. A remote central monitoring unit generates analytical status data from the data indicative of physiological parameters, the derived data, and analytical status data that has previously been generated. A data storage device stores the data received and generated by the central monitoring unit. The system includes means for establishing electronic communication between the sensor device and the central monitoring unit, and means for transmitting the data to a recipient. The sensor device may also generate data indicative of contextual parameters of the individual, which may then be used when generating the analytical status data. The system may be used for monitoring and providing feedback relating to the degree to which an individual has followed a suggested routine.

In another prior art solution, provided are means and methods for measuring heat flux between a living body and an ambient atmosphere and for generating information related thereto. Means include a heat pipe configuration comprising at least one heat conduit in thermal communication with a heat flux sensor and a surface of a living body. Methods include employing such a heat pipe configuration. Heat pipe configuration may be used in a device included in a system for detecting, monitoring and reporting human physiological information.

In yet another prior art solution, provided is a system for monitoring and reporting the activity level and caloric expenditure of an individual. The system has sensors in electronic communication with a wearable device. A processor is programmed to communicate with the sensor and a computing device to activity data from movement-related data. A remote server receiving the activity data and the data from computing device, and generates a graphical presentation. The graphical presentation comprises information related to the activity data. The wearable device also provides visual and audio data.

In yet another prior art solution, disclosed is a method and system for monitoring a health condition of an individual. At least one physiological parameter of the individual is measured and measured data is then processed in order to detect a potential danger in the measured parameter. Upon detecting the potential danger, a warning signal representative of the potential danger is generated, and a signal for indicating to the individual to actuate a personal communication unit is provided during a predetermined period of time. Upon actuating the personal communication unit, an information message representative of the warning signal is transmitted to the individual. In the absence of actuating the personal communication unit during the predetermined period of time, data indicative of the warning signal is transmitted to a central monitoring station.

However, none of the aforementioned prior art solutions enable to substantially overcome the various drawbacks associated with conventional techniques for monitoring health of individuals.

Therefore, in light of the above discussion, there is a need to overcome various drawbacks associated with conventional techniques for monitoring health of individuals and provide improved healthcare monitoring systems and methods.

### SUMMARY

This section provides a general summary of the disclosure, and is not a comprehensive disclosure of its full scope or all of its features.

The present disclosure seeks to provide an integrated healthcare monitoring system and a method thereof.

According to another aspect, an embodiment of the present disclosure provides an integrated healthcare monitoring method, the method comprising: accessing one or more information pertaining to a member from a member record database; analysing said one or more information in respect of one or more attributes comprising: absences from work, performance reviews, relationship map, claims relating to behavioural issue, log-in hours, family history, age group, gender, performance improvement programs, prior work area, maternity period in case of females, social media behaviour, address, marital status, compensation, salary grade, skillset map, and current job profile; generating a first set of questionnaire to assess mental health of the member based on analysis said one or more information; generating a first score based on a response received from the member in respect of the first set of questionnaire, and a second score in respect of each of the one or more attributes based on analysis of said one or more information; mapping the first score and second score respectively with a list of pre-defined scores in a pre-created matrix, said pre-created matrix including a mapping of one or more behavioural health information corresponding to a pre-assigned score, said one or more behavioural health information including at least one of severity level of mental health and one or more mental health underlying issues; accessing and processing data relating to at least one of genetics, microbiome tests of the member to identify one or more mental health underlying issues of the member; ascertaining if the one or more mental health underlying issues identified from said mapping correlate with the mental health underlying issues identified from processing of the data relating to the at least one of genetics, microbiome tests of the member, and recommending one or more treatment processes of a first type to be followed for restoration of the mental wellness of the member on basis the one or more mental health underlying issues confirmed on a positive ascertaining.

According to another aspect, an embodiment of the present disclosure provides an integrated health care monitoring system, the system comprising: a user device communicatively coupled to at least one healthcare providers, wherein the user device is configured to access information, pertaining to a health of a member, from the at least one health providers, and wherein the information comprises electronic health records of the member pertaining to one or more of one or more diagnostics, therapeutics, key symptoms, signs, complications, or outcomes of a disease; a member record database communicatively coupled to a member information portal and the user device, wherein the member record database is configured to: receive, from the member information portal, information pertaining to demographic data comprising age, sex, geographical location, socio-economic classification, job classification, job performance data, absenteeism caused due to physical and mental health issues; receive, from the user device, the information accessed from the at least one health providers, pertaining to the health of the member; a member wellness artificial intelligence engine communicatively coupled to the member record database and one or more medical-based databases, wherein the member wellness artificial intelligence engine is configured to: access, from the member record database, the information pertaining to the health of the member; receive member-specific information from the one or more medical-based databases, wherein the member-specific information is relevant to the health of the member; receive member genome data and member biome data from the at least one healthcare providers; process the information received from the member record database, the received member genome data, the received member biome data, the information received from the medical-based databases to generate a propensity assessment for the member, wherein the propensity assessment comprises a probabilistic measure of future physical risks and mental health risks that the member is prone to; a member results suite communicatively coupled to the member wellness artificial intelligence engine, the member results suite configured to: process the propensity assessment to generate one or more recommendations for the member based on the physical risks and the mental risks that the member is prone to; present the generated one or more recommendations on the user device under one of categories: no touch process, low touch process, and high touch process, wherein: —the no touch process comprises one or more recommendations relating to at least one of sleep, mindfulness, physical activity, supplements, journaling including diet; —the low touch process comprises one or more recommendations via an assigned healthcare professional; —the high touch process comprises one or more recommendations via at least one doctors.

In another aspect, an embodiment of the present disclosure provides an integrated healthcare monitoring method, the method comprising: accessing information, pertaining to a health of a member via a user device, from at least one health providers, and wherein the information comprises electronic health records of the member pertaining to one or more of one or more diagnostics, therapeutics, key symptoms, signs, complications, or outcomes of a disease; receiving information pertaining to demographic data comprising age, sex, geographical location, socio-economic classification, job classification, job performance data, absenteeism caused due to physical and mental health issues from the member information portal; receiving the information accessed from the at least one health providers, pertaining to the health of the member from the user device; storing the received information in a member record database; accessing the information pertaining to the health of the member from the member record database; receiving member-specific information from one or more medical-based databases, wherein the member-specific information is relevant to the health of the member; receiving member genome data and member biome data from the at least one healthcare providers; processing the information received from the member record database, the received member genome data, the received member biome data, the information received from the medical-based databases to generate a propensity assessment for the member via a member wellness artificial intelligence engine, wherein the propensity assessment comprises a probabilistic measure of future physical risks and mental health risks that the member is prone to; processing the propensity assessment to generate one or more recommendations for the member based on the physical risks and the mental risks that the member is prone to via the member results suite; presenting the generated one or more recommendations on the user device under one of categories: no touch process, low touch process, and high touch process, wherein: —the no touch process comprises one or more recommendations relating to at least one of sleep, mindfulness, physical activity, supplements, journaling including diet; —the low touch process comprises one or more recommendations via an assigned healthcare professional; —the high touch process comprises one or more recommendations via at least one doctors.

Further areas of applicability will become apparent from the description provided herein. The description and specific examples in this summary are intended for purposes of illustration only and are not intended to limit the scope of the present disclosure.

### DESCRIPTION OF THE DRAWINGS

The summary above, as well as the following detailed description of illustrative embodiments, is better understood when read in conjunction with the appended drawings.

For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram of an integrated healthcare monitoring system, in accordance with an embodiment of the present disclosure;

FIG. 2 is a schematic diagram of an exemplary environment of components of information in a member record database, in accordance with an embodiment of the present disclosure;

FIG. 3 is a schematic diagram of an exemplary environment of components of feeding data to member wellness artificial intelligence engine, in accordance with an embodiment of the present disclosure;

FIG. 4 is a schematic diagram of an exemplary environment depicting components of a member results suite, in accordance with an embodiment of the present disclosure;

FIGS. 5A-B, there is shown a flowchart illustrating steps of an integrated healthcare monitoring method, in accordance with an embodiment of the present disclosure;

FIGS. 6A-B, there is shown a flowchart illustrating steps of an integrated healthcare monitoring method, in accordance with another embodiment of the present disclosure; and

FIG. 7 is a block diagram of an integrated healthcare monitoring system, in accordance with another embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DESCRIPTION OF EMBODIMENTS

Exemplary embodiments will now be described more fully with reference to the accompanying drawings.

The exemplary embodiments are provided so that this disclosure will be thorough, and will fully convey the scope to persons skilled in the art. Numerous specific details are set forth such as examples of specific components, devices, and methods, to provide a thorough understanding of embodiments of the present disclosure. It will be appreciated by persons skilled in the art that specific details need not be employed. Exemplary embodiments may be embodied in many different forms. Thus, neither should be construed to limit the scope of the disclosure. In some example embodiments, well-known processes, well-known device structures, and well-known technologies are not described in detail.

The terminology used herein is for the purpose of describing particular exemplary embodiments only and is not intended to be limiting. As used herein, singular forms such as “a,” “an,” and “the” may be intended to include corresponding plural forms as well, unless the context clearly indicates otherwise. Furthermore, terms akin to “comprises,” “comprising,” “including,” and “having,” are inclusive and therefore, specify the presence of stated features, integers, steps, operations, elements, and/or components, but do not preclude the presence or addition of one or more other features, integers, steps, operations, elements, components, and/or groups thereof.

When an element or layer is referred to as being “on,” “engaged to,” “connected to,” or “coupled to” another element or layer, it may be disposed directly on, engaged, connected or coupled to the other element or layer, or intervening elements or layers may be present therein. However, when an element is referred to as being “directly on,” “directly engaged to,” “directly connected to,” or “directly coupled to” another element or layer, there may be no intervening elements or layers present. Other words used to describe a relationship between elements should be interpreted in a like manner (e.g., “between” versus “directly between,” “adjacent” versus “directly adjacent,” etc.). As used herein, the term “and/or” includes any and all combinations of one or more of the associated listed items.

Spatially relative terms such as “inner,” “outer,” “beneath,” “below,” “lower,” “above,” “upper,” and the like may be used herein for ease of description, to describe an element's or a feature's relationship to another element(s) or feature(s) as illustrated in the figures. Furthermore, spatially relative terms may be intended to encompass different orientations of the device in use or in operation, in addition to one or more orientations depicted in the figures. For example, if the device in the figures is turned over, elements described as “below” or “beneath” other elements or features would then be oriented “above” the other elements or features. Thus, the example term “below” can encompass both an orientation of above and below. It will be appreciated that the device may be otherwise oriented (rotated 90 degrees or at other orientations) and the spatially relative descriptors used herein should be interpreted accordingly.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Moreover, if any method steps, processes, and operations are described, they are not to be construed as necessarily requiring their performance in the particular order discussed or illustrated, unless specifically identified as an order of performance. It is also to be understood that additional or alternative steps may be employed.

According to an aspect, an embodiment of the present disclosure provides an integrated health care monitoring system, the system comprising:
a user device communicatively coupled to at least one healthcare providers, wherein the user device is configured to access information, pertaining to a health of a member, from the at least one health providers, and wherein the information comprises electronic health records of the member pertaining to one or more of one or more diagnostics, therapeutics, key symptoms, signs, complications, or outcomes of a disease;
a member record database communicatively coupled to a member information portal and the user device, wherein the member record database is configured to:
   receive, from the member information portal, information pertaining to demographic data comprising age, sex, geographical location, socio-economic classification, job classification, job performance data, absenteeism caused due to physical and mental health issues; and
   receive, from the user device, the information accessed from the at least one health providers, pertaining to the health of the member;
a member wellness artificial intelligence engine communicatively coupled to the member record database and one or more medical-based databases, wherein the member wellness artificial intelligence engine is configured to:
   access, from the member record database, the information pertaining to the health of the member;
   receive member-specific information from the one or more medical-based databases, wherein the member-specific information is relevant to the health of the member;
   receive member genome data and member biome data from the at least one healthcare providers; and
   process the information received from the member record database, the received member genome data, the received member biome data, the information received from the medical-based databases to generate a propensity assessment for the member, wherein the propensity assessment comprises a probabilistic measure of future physical risks and mental health risks that the member is prone to; and
a member results suite communicatively coupled to the member wellness artificial intelligence engine, the member results suite configured to:
   process the propensity assessment to generate one or more recommendations for the member based on the physical risks and the mental risks that the member is prone to;
   present the generated one or more recommendations on the user device under one of categories: no touch process, low touch process, and high touch process, wherein:
      the no touch process comprises one or more recommendations relating to at least one of sleep, mindfulness, physical activity, supplements, journaling including diet;
      the low touch process comprises one or more recommendations via an assigned healthcare professional;
      the high touch process comprises one or more recommendations via at least one doctors.

According to another aspect, an embodiment of the present disclosure provides an integrated healthcare monitoring method, the method comprising:
accessing information, pertaining to a health of a member via a user device, from at least one health providers, and wherein the information comprises electronic health records of the member pertaining to one or more of one or more diagnostics, therapeutics, key symptoms, signs, complications, or outcomes of a disease;
receiving information pertaining to demographic data comprising age, sex, geographical location, socio-economic classification, job classification, job performance data, absenteeism caused due to physical and mental health issues from the member information portal;
receiving the information accessed from the at least one health providers, pertaining to the health of the member from the user device;
storing the received information in a member record database;
accessing the information pertaining to the health of the member from the member record database;
receiving member-specific information from one or more medical-based databases, wherein the member-specific information is relevant to the health of the member;
receiving member genome data and member biome data from the at least one healthcare providers;
processing the information received from the member record database, the received member genome data, the received member biome data, the information received from the medical-based databases to generate a propensity assessment for the member via a member wellness artificial intelligence engine, wherein the propensity assessment comprises a probabilistic measure of future physical risks and mental health risks that the member is prone to;
processing the propensity assessment to generate one or more recommendations for the member based on the physical risks and the mental risks that the member is prone to via the member results suite; and
presenting the generated one or more recommendations on the user device under one of categories: no touch process, low touch process, and high touch process, wherein:
   the no touch process comprises one or more recommendations relating to at least one of sleep, mindfulness, physical activity, supplements, journaling including diet;
   the low touch process comprises one or more recommendations via an assigned healthcare professional; and
   the high touch process comprises one or more recommendations via at least one doctors.

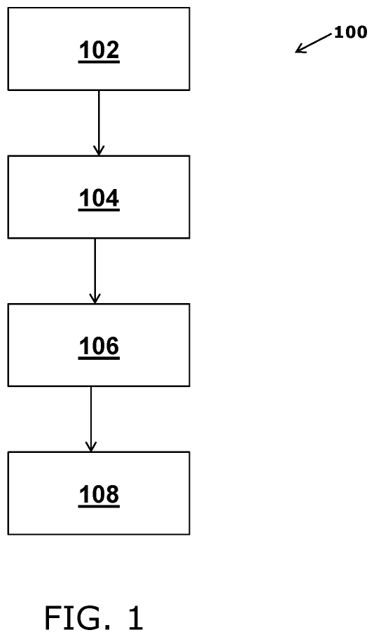

Referring to FIG. 1, there is shown a block diagram of an integrated healthcare monitoring system 100, in accordance with an embodiment of the present disclosure. As shown, the integrated health monitoring system 100 comprises a user device 102, a member record database 104, a member wellness artificial intelligence engine 106, and a member results suite 108.

The user device 102 is communicatively coupled to at least one healthcare provider. The user device 102 is configured to access information, pertaining to a health of a member, from the at least one health providers, and wherein the information comprises electronic health records of the member pertaining to one or more of one or more diagnostics, therapeutics, key symptoms, signs, complications, or outcomes of a disease. Throughout the present disclosure, the term “member” as used herein refers to a person or a human being whose health is monitored using the system. Optionally, the person is required to register in the system 100 via the user device 102 to be the member enrolled in the system 100. Throughout the present disclosure, the term “healthcare provider” is used broadly and inclusively herein to refer to the medical, clerical, office staff, or other employees, contractors, associates, organizations, or other persons associated with the healthcare provider and otherwise involved with the medical treatment or medical assistance of the member.

Throughout the present disclosure, the term “user device” as used herein refers to an electronic device associated with (or used by) a user that is capable of enabling the user to perform specific tasks associated with the application portal. Furthermore, the user equipment is intended to be broadly interpreted to include any electronic device that may be used for voice and/or data communication over a wireless communication network. The user devices can include and is not limited to a, mobile phones, smart telephones, Mobile Internet Devices (MIDs), tablet computers, Ultra-Mobile Personal Computers (UMPCs), Personal Digital Assistants (PDAs), web pads, Personal Computers (PCs), Handheld PCs, laptop computers, desktop computers, Network-Attached Storage (NAS) devices, large-sized touch screens with embedded PCs, and interactive entertainment devices, such as game consoles, Television (TV) sets and Set-Top Boxes (STBs), a video slot machine, a video poker machine, a kiosk, a casino personal device, and typically includes many or all of the elements described above relative to the integrated health monitoring system.

Optionally, the system 100 further comprises a user interface (not shown) pertaining to the user device 102. The user device 102 is configured to receive information from the member, pertaining to a physical health, a mental health, and a social health of the member. It will be appreciated that in a first step the member is required to register with the system 100 by entering personal information such as a name, address, contact details, and so forth via the user interface pertaining to the user device 102.

Throughout the present disclosure the term “user interface” as used herein refers to a structured set of user interface elements rendered on a display screen associated with the user device. Optionally, the user interface (UI) rendered on the display screen is generated by any collection or set of instructions executable by an associated digital system. Additionally, the user interface (UI) is operable to interact with the users to convey graphical and/or textual information and receive input from the user. Specifically, the user interface (UI) used herein is a graphical user interface (GUI). Furthermore, the user interface (UI) elements refer to visual objects that have a size and position in user interface (UI). A user interface element may be visible, though there may be times when a user interface element is hidden. A user interface control is considered to be a user interface element. Text blocks, labels, text boxes, list boxes, lines, and images windows, dialog boxes, frames, panels, menus, buttons, icons, etc. are examples of user interface elements. In addition to size and position, a user interface element may have other properties, such as a margin, spacing, or the like. Beneficially, the user interface facilitates user interaction in the gaming applications within the integrated health monitoring system.

Optionally, the user device 102 is configured to present health and wellness survey. Further, the user device 102 is configured to receive information from the member pertaining to a physical health, a mental health, and a social health of the member. Notably, the member provides answers to the health and wellness surveys and information related to a health of the member may be extracted from one or more answers provided by the member, in response to the surveys provided.

The system 100 comprises a member record database 104 communicatively coupled to a member information portal and the user device. Throughout the present disclosure the term “member record database” as used herein refers to used herein refers to an organized body of digital information regardless of the manner in which the data or the organized body thereof is represented. Optionally, the database may be software, firmware and/or any combination thereof. For example, the organized body of related data may be in the form of a table, a map, a grid, a packet, a datagram, a file, a document, a list or in any other form. The database includes any data storage software and systems, such as, for example, a relational database like IBM DB2 and Oracle 9. Optionally, the database may be used interchangeably herein as database management system, as is common in the art. Furthermore, the database management system refers to the software program for creating and managing one or more databases. Optionally, the database may be operable to supports relational operations, regardless of whether it enforces strict adherence to the relational model, as understood by those of ordinary skill in the art.

Throughout the present disclosure, the term “member information portal” refers to a software component for collection of information related to the member. Optionally, the member information portal is configured to analyze one or more attributes pertaining to a member to generate the information, and wherein the one or more attributes comprise: absences from work, performance reviews, relationship map, claims relating to behavioral issue, location of work, log-in hours, family history, age group, gender, performance improvement programs, prior work area, maternity period in case of females, social media behavior, address, marital status, compensation, salary grade, skillset map, and current job profile. In an example, the member information portal is a HR portal of the member.

Further, the member record database 104 is configured to receive, from the member information portal, information pertaining to demographic data comprising age, sex, geographical location, socio-economic classification, job classification, job performance data, absenteeism caused due to physical and mental health issues. The member record database 104 is configured to receive, from the user device, the information accessed from the at least one health providers, pertaining to the health of the member. Notably, the member record database 104 is configured to store information related to a number of members in a structured format.

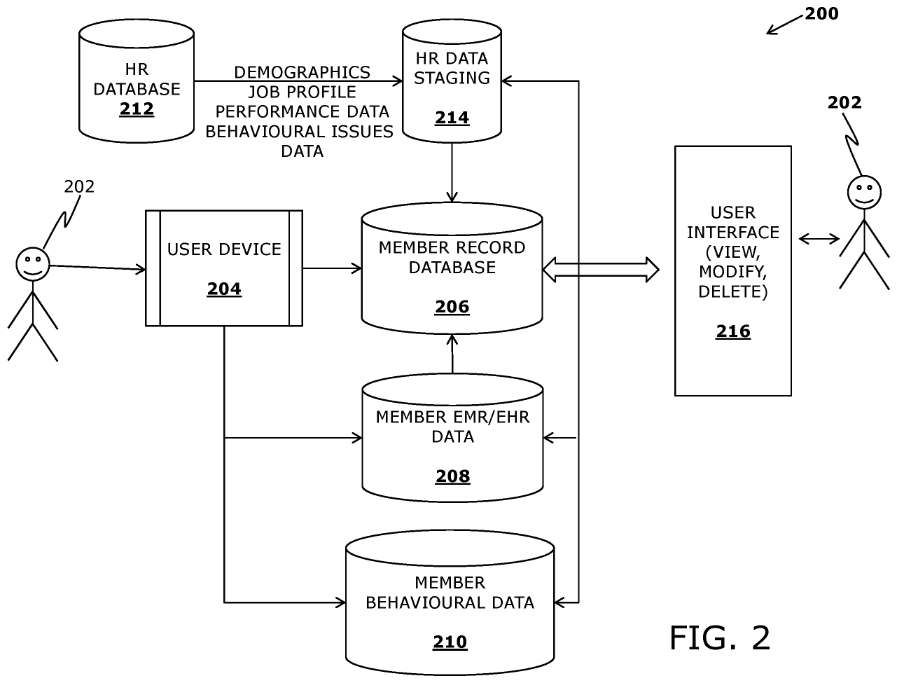

Referring to FIG. 2, there is shown an exemplary environment of components 200 of information in a member record database, in accordance with an embodiment of the present disclosure. As shown, the user 202 is allowed to register as a member in the system via the user device 204. Herein, the user device 204 is communicatively coupled to the member record database 206. In particular, the user 202 is allowed to store information pertaining to health in the member record database 206 via the user device 204. Further, the member record database 206 receives data from member EMR/EHR data 208 and behavioral data 210, in combination with HR data 212 and HR staging data 214 into the member record database 206. Further, as shown, the user 202 is allowed to access the member record database 206 via the user interface 216. In particular, the user 202 is allowed to view, delete and modify health information as and when required.

Referring back to FIG. 1, as shown, the system comprises a member wellness artificial intelligence engine 106. The member wellness artificial intelligence engine 106 is communicatively coupled to the member record database 104 and one or more medical-based databases. The member wellness artificial intelligence engine 106 is configured to access, from the member record database 104, the information pertaining to the health of the member. Further, the member wellness artificial intelligence engine 106 is configured to receive member-specific information from the one or more medical-based databases (not shown), wherein the member-specific information is relevant to the health of the member. Further, the member wellness artificial intelligence engine 106 is configured to receive member genome data and member biome data from the at least one healthcare provider. Further, the member wellness artificial intelligence engine 106 is configured to process the information received from the member record database 104, the received member genome data, the received member biome data, the information received from the medical-based databases to generate a propensity assessment for the member. Herein, the propensity assessment comprises a probabilistic measure of future physical risks and mental health risks that the member is prone to.

Herein the member wellness artificial intelligence system 106 is a processing unit employing artificial intelligence algorithm to determine the aforesaid results. Throughout the present disclosure the term “processing unit” refers to a computational element that is operable to respond to and processes instructions that drive the system. Optionally, the processor includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processor unit” may refer to one or more individual processors, processing devices and various elements associated with a processing device that may be shared by other processing devices. Additionally, the one or more individual processors, processing devices and elements are arranged in various architectures for responding to and processing the instructions that drive the system.

Throughout the present disclosure, the term “artificial intelligence” as used herein refers to any mechanism or computationally intelligent system that combines knowledge, techniques, and methodologies for controlling a bot, such as the first digital entity or other element within a computing environment. Furthermore, the artificial intelligence (A) is configured to apply knowledge and that can adapt it-self and learn to do better in changing environments. Additionally, employing any computationally intelligent technique, the artificial intelligence (AI) is operable to adapt to unknown or changing environment for better performance. The artificial intelligence (AI) includes fuzzy logic engines, decision-making engines, pre-set targeting accuracy levels, and/or programmatically intelligent software. The artificial intelligence (AI) in the context of the present disclosure relates to software-based algorithms that are executable upon computing hardware and are operable to adapt and adjust their operating parameters in an adaptive manner depending upon information that is presented to the software-based algorithms when executed upon the computing hardware. Optionally, artificial intelligence (AI) employ any one or combination of the following computational techniques: constraint program, fuzzy logic, classification, conventional artificial intelligence, symbolic manipulation, fuzzy set theory, evolutionary computation, cybernetics, data mining, approximate reasoning, derivative-free optimization, decision trees, or soft computing.

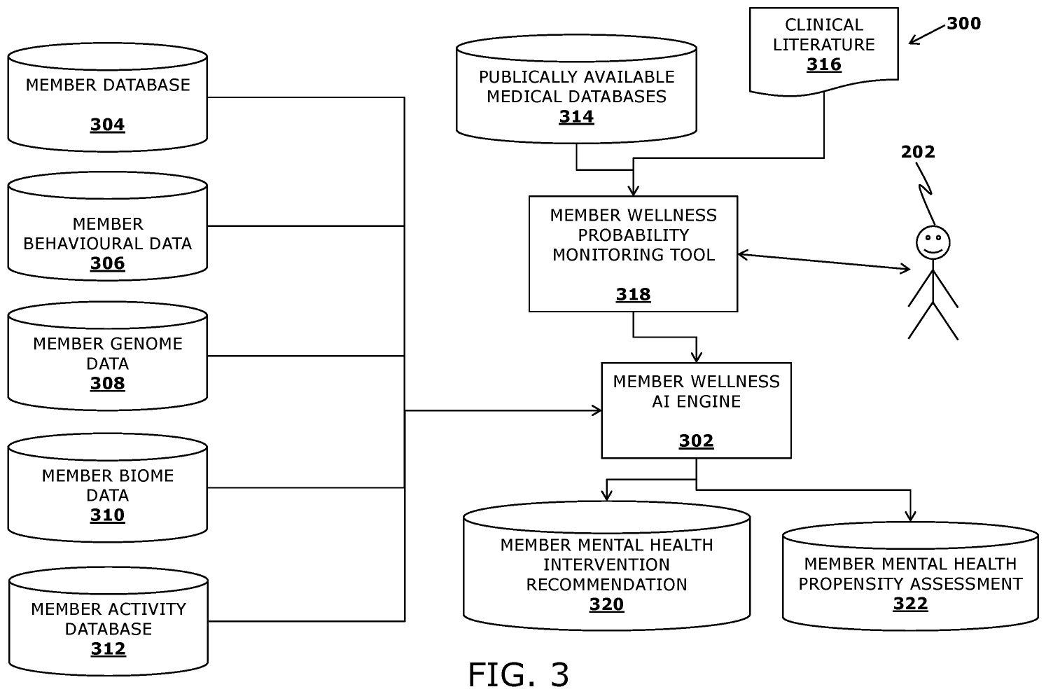

Referring to FIG. 3, there is shown an exemplary environment of components 300 of feeding data to member wellness artificial intelligence engine 302, in accordance with an embodiment of the present disclosure. As shown, the member wellness artificial intelligence engine 302 receives information from member database 304 comprising health information, member behavioral data 306, member genome data 308, and member biome data 310. Further, the member wellness artificial intelligence engine 302 receives data from member activity database 312 comprising data recorded in response to one or more activities by the member. Further, a member wellness probability monitoring tool 318 receives data from publicly available medical databases 314 and clinical literature 316, comprising information around known practices in medical science, and further performs analysis. Further, the member wellness probability monitoring tool 318 receives information from the member 202. Herein, the member wellness artificial intelligence engine 302 receives data from the member wellness probability monitoring tool 318 to determine member mental health intervention recommendation 320 and member mental health propensity assessment 322 comprising one or more recommendations for the member 202.

Now referring back to FIG. 1, as shown, the system 100 comprising a member results suite 108 communicatively coupled to the member wellness artificial intelligence engine 106.

Herein, the member results suite configured to process the propensity assessment to generate one or more recommendations for the member based on the physical risks and the mental risks that the member is prone to. Optionally, the one or more recommendations comprise one or more of: targeted dietary changes and supplements, option to purchase targeted meals from one the integrated platforms, specify physical fitness activity regimen like to regulate specific condition, option to subscribe to one or more of integrated fitness platforms, improve engagement to create and align groups of members with common goals, interests or diet and/or physical fitness regimen, tele-medicine consultation with doctors on the targeted plan follow-ups, food and exercise log. Furthermore, optionally, the system is integrated with third party platforms, wherein the third party platforms comprise at least one of: Gyms, health monitoring applications, health monitoring smart watches, online shopping stores, pharmaceuticals and drug delivery applications, social networking applications.

Further, the member results suite 108 is configured to present the generated one or more recommendations on the user device 102 under one of categories: no touch process, low touch process, and high touch process. Notably, the no touch process comprises one or more recommendations relating to at least one of sleep, mindfulness, physical activity, supplements, journaling including diet; the low touch process comprises one or more recommendations via an assigned healthcare professional; and the high touch process comprises one or more recommendations via at least one doctors. Optionally, the member results suite 108 is further configured to trigger an early intervention alarm via the user device 102, wherein the alarm is triggered when a health condition of the member is above a predefined critical condition.

According to an embodiment, the integrated health care monitoring system 100 further comprises a member activity database (not shown) configured to record actions of the member based on the one or more recommendations, Herein, the actions of the member are recorded via one or more bio-sensors. Herein, the one or more bio-sensors may be health tracking devices such as smart watches that are in contact with the body and continuously measure vitals of the person. Further, the member activity database is configured to update the member wellness artificial engine 106 based on the recorded actions of the member in order to update the one or more recommendations generated for the member. It will be appreciated that as a health of member changes over a period of time, the one or more recommendations are also modified accordingly.

Optionally, the member results suite is further configured to share details of a progress in health of a first member with a second member via the user device 102. In an example, a member can share details on progress of their health with a friend, a family member, or a spouse such that the second member is allowed to view the progress of the first member via the user interface. Further, the member results suite is configured to schedule and synchronize one or more common activities for the first member and the second member. Notably, one or more common activities such as following a similar diet, registering for a common gym, and the like can be synchronized for the first member and the second member. Further, the member results suite 108 is configured to assign wellness points to the first member and the second member, show avatars for the first member and the second member. Notably, the wellness points are assigned to the members in order to motivate them to lead a more healthy life. Further, the member results suite 108 is configured to compare a progress of the first member to the second member via the user device 102.

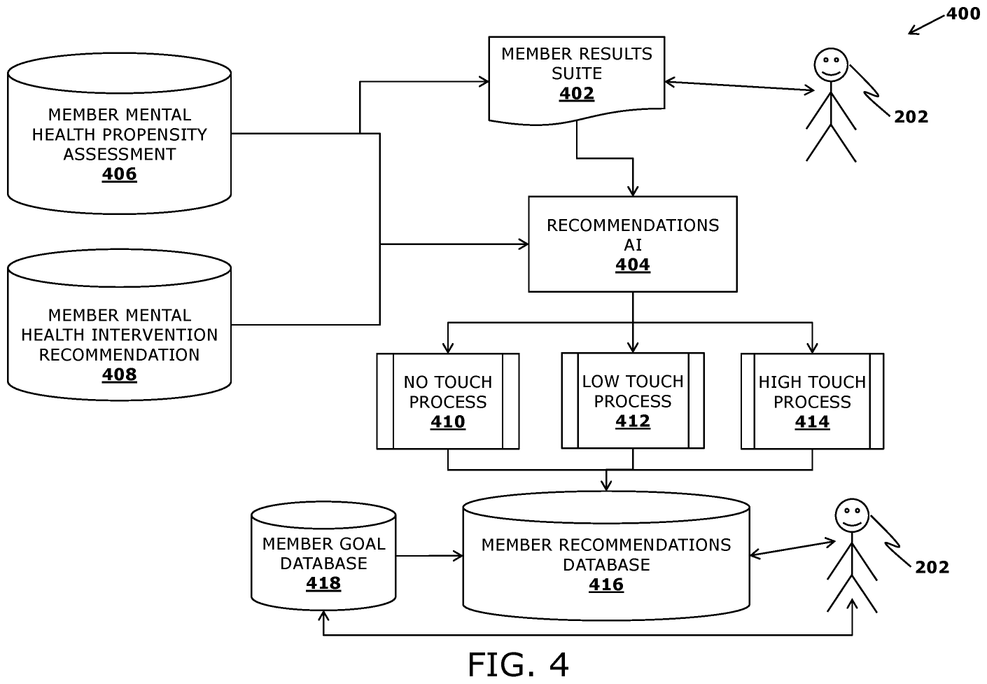

Referring to FIG. 4, there is shown an exemplary environment depicting components 400 of a member results suite, in accordance with an embodiment of the present disclosure. As shown, the member results suite 402 receives information from member mental health propensity assessment 406 and member mental health intervention recommendation 408 to provide one or more recommendations via recommendations AI. Further, recommendations AI 404 generate no touch process 410, low touch process 412 and high touch process 414 (as explained above in FIG. 1). The recommendations AI 404 generates one amongst no touch process 410, low touch process 412 and high touch process 414 respectively at a given instance based on the information received from the from member mental health propensity assessment 406 and member mental health intervention recommendation 408. Further, the one or more recommendations are stored in member recommendation database 416 communicatively coupled with a member goal database 418 comprising targets of the member 202.

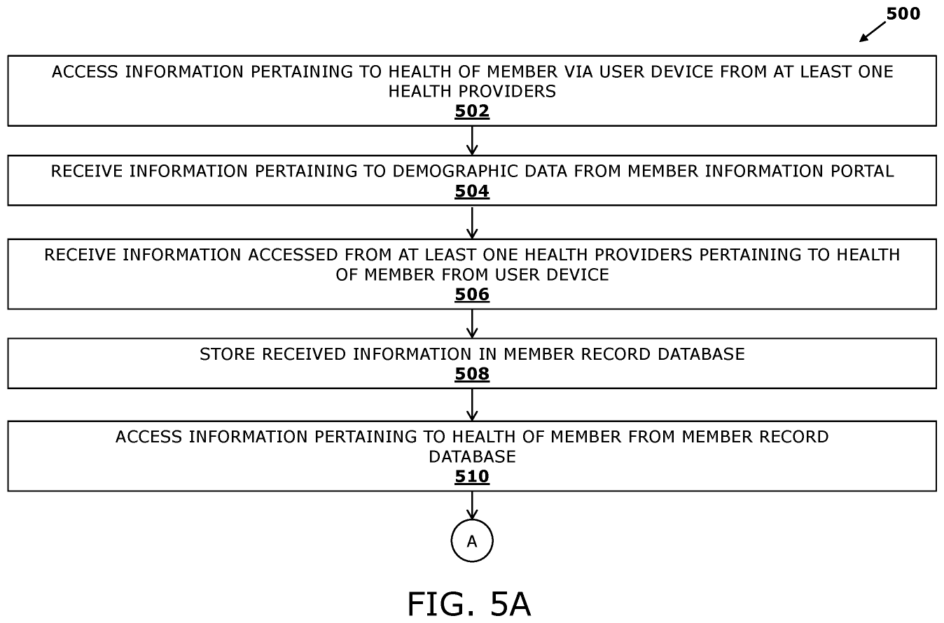

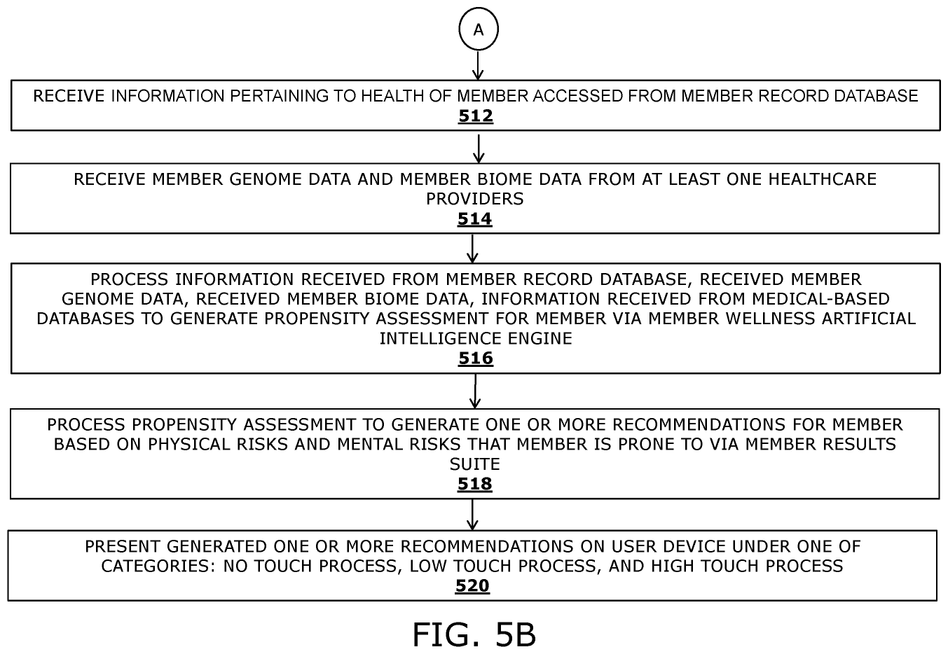

Referring to FIGS. 5A-B, there is shown a flowchart illustrating steps of an integrated healthcare monitoring method 500, in accordance with an embodiment of the present disclosure. At a step 502, information pertaining to a health of a member is accessed via a user device from at least one health providers. The information comprises electronic health records of the member pertaining to one or more of one or more diagnostics, therapeutics, key symptoms, signs, complications, or outcomes of a disease. At a step 504, information pertaining to demographic data is received from the member information portal, the information comprising age, sex, geographical location, socio-economic classification, job classification, job performance data, absenteeism caused due to physical and mental health issues. At a step 506, the information accessed from the at least one health providers pertaining to the health of the member is received from the user device. At a step 508, the received information is stored in a member record database. At a step 510, the information pertaining to the health of the member is accessed from the member record database. At a step 512, member-specific information is received from one or more medical-based databases. The member-specific information is relevant to the health of the member. At a step 514, member genome data and member biome data are received from the at least one healthcare providers. At a step 516, the information received from the member record database, the received member genome data, the received member biome data, the information received from the medical-based databases are processed to generate a propensity assessment for the member via a member wellness artificial intelligence engine. The propensity assessment comprises a probabilistic measure of future physical risks and mental health risks that the member is prone to. At a step 518, the propensity assessment is processed to generate one or more recommendations for the member based on the physical risks and the mental risks that the member is prone to via the member results suite. At a step 520, the generated one or more recommendations are presented on the user device under one of categories: no touch process, low touch process, and high touch process. The no touch process comprises one or more recommendations relating to at least one of sleep, mindfulness, physical activity, supplements, journaling including diet. The low touch process comprises one or more recommendations via an assigned healthcare professional. The high touch process comprises one or more recommendations via at least one doctors.

In an embodiment, the healthcare monitoring method 500 further comprises recording actions of the member based on the one or more recommendations, wherein the actions of the member are recorded via one or more bio-sensors; and updating the member wellness artificial engine based on the recorded actions of the member in order to update the one or more recommendations generated for the member. In an embodiment, the one or more recommendations comprise one or more of: targeted dietary changes and supplements, option to purchase targeted meals from one the integrated platforms, specify physical fitness activity regimen like to regulate specific condition, option to subscribe to one or more of integrated fitness platforms, improve engagement to create and align groups of members with common goals, interests or diet and/or physical fitness regimen, tele-medicine consultation with doctors on the targeted plan follow-ups, food and exercise log.

In an embodiment, the healthcare monitoring method 500 further comprises, triggering, by the member results suite, to an early intervention alarm via the user device, wherein the alarm is triggered when a health condition of the member is above a predefined critical condition.

In an embodiment, the healthcare monitoring method 500 further comprises analysing one or more attributes pertaining to a members to generate the information, and wherein the one or more attributes comprise: absences from work, performance reviews, relationship map, claims relating to behavioural issue, location of work, log-in hours, family history, age group, gender, performance improvement programs, prior work area, maternity period in case of females, social media behaviour, address, marital status, compensation, salary grade, skillset map, and current job profile.

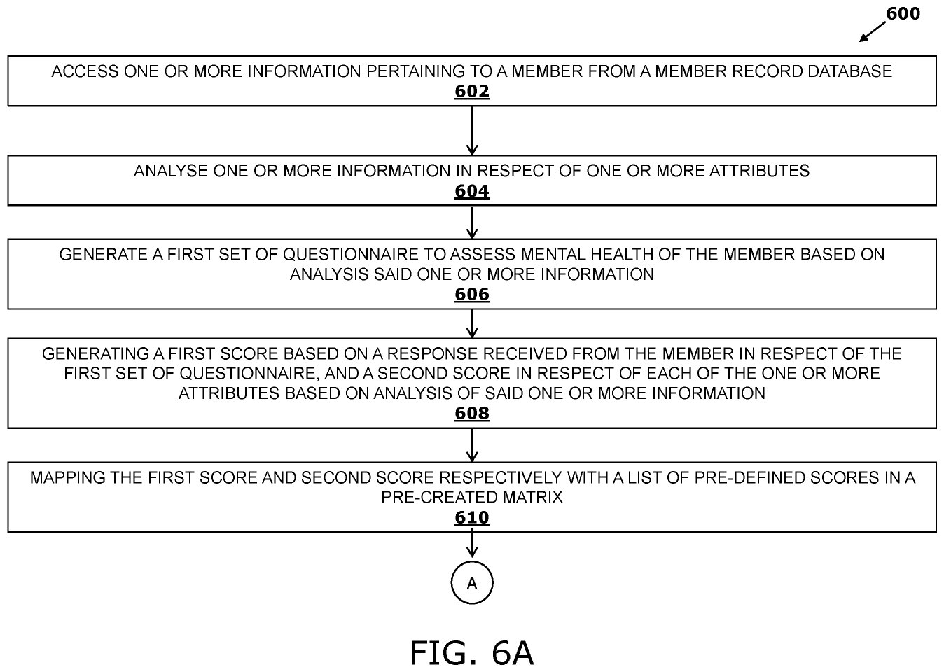

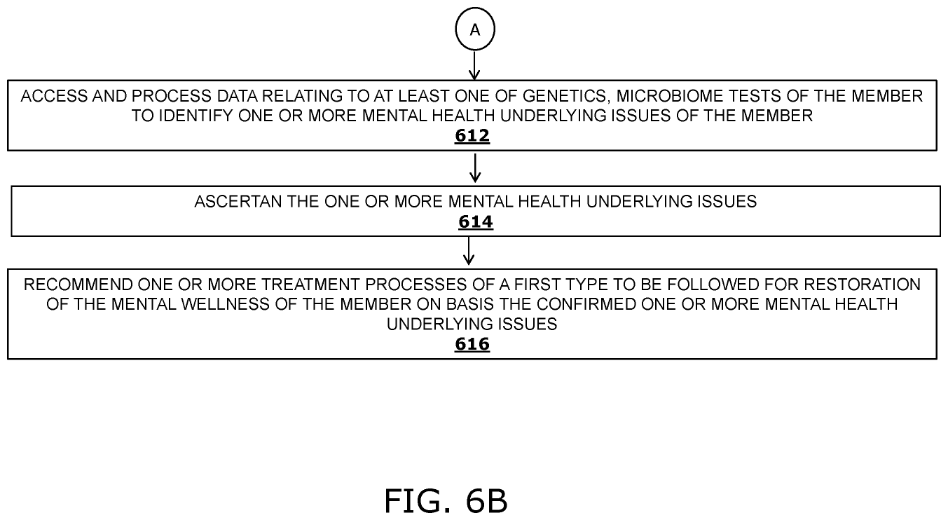

Referring to FIG. 6, there is shown a flowchart illustrating steps of an integrated healthcare monitoring method 600, in accordance with another embodiment of the present disclosure. At a step 602, one or more information pertaining to a member from a member record database is accessed. The one or more information may relate to one or more attributes comprising: absences from work, performance reviews, relationship map, claims relating to behavioural issue, log-in hours, family history, age group, gender, performance improvement programs, prior work area, maternity period in case of females, social media behaviour, address, marital status, compensation, salary grade, skillset map, and current job profile. In an exemplary implementation, the one or more information may be created using the data available from the employment records. The employment data is stored in the member record database, and is used for identifying the mental state of the member. At a step 604, the one or more information in respect of one or more attributes is analysed to determine the mental state of the member. A predefined database is created after analysis of a plurality of members/users and their employee attributes and their mental state levels are accordingly mapped. Based on the aforesaid predefined database, the members' specific mental state may be assessed. In an implementation, the one or more attributes are assigned weightages to determine the appropriate mental health condition of the member. For instance, absences from work, performance reviews, may be given higher weightage over is compensation or salary grade to assess the mental health condition/behaviour of a member. At a step 606, a first set of questionnaire to assess mental health of the member based on analysis the one or more information is generated. The first set of questions may be fetched from a pre-stored database. The first set of questions stored in the pre-defined database is chosen selectively to determine the mental state/behaviour of a member. The first set of questions contains one or more questions that help in ascertain the attributes relating to mental health of the user. The attributed may relate to how a member feels about itself, emotion levels, personal relationships, comfort level with other people, happiness levels, ability to feel, express and manage a range of positive and negative emotions, ability to cope with and manage change and uncertainty to name a few. At a step 608, a first score based on a response received from the member in respect of the first set of questionnaire, and a second score in respect of each of the one or more attributes based on analysis of said one or more information is generated and assigned. At a step 610, the first score and second score respectively are mapped with a list of pre-defined scores in a pre-created matrix, said pre-created matrix including a mapping of one or more behavioural health information corresponding to a pre-assigned score, said one or more behavioural health information including at least one of severity level of mental health and one or more mental health underlying issues. Further, the method includes a step 612 of accessing and processing data relating to at least one of genetics, microbiome tests of the member to identify one or more mental health underlying issues of the member. This processing is done to confirm that the underlying mental health issues are correct and to adjudicate correctly upon the root cause of the improper/unstable mental health of the member. At a step 614, it is ascertained if the one or more mental health underlying issues identified from said mapping correlate with the mental health underlying issues identified from processing of the data relating to the at least one of genetics, microbiome tests of the member. Once the underlying mental health issues and the root cause of those issues are confirmed, one or more treatment processes of a first type are recommended at a step 616. The treatment process is to be followed for restoration of the mental wellness of the member on basis the one or more mental health underlying issues confirmed on a positive ascertaining.

In another embodiment, the integrated healthcare monitoring method 600 includes accessing and processing data relating to at least one of lab data including blood markers, allergy information, RNA, plasma, DNA, SNPs; and activity data of the member for ascertaining if the one or more mental health underlying issues identified from said mapping correlate with the mental health underlying issues identified from processing of the data relating to the at least one of genetics, microbiome tests of the member. The lab data is generally referred to adjudicate upon further root causes of the mental health of member.

In another embodiment, the integrated healthcare monitoring method 600 includes accessing and processing data from one or more data from one or more biosensors configured in wearable smart device worn by the member, wherein the data from the biosensors includes data relating to breathe and oxygen levels, heart beat level, glucose, stress levels, mood, anxiety levels. The smart devices may include smart watches, smart glass, smart necklace, smart wrist band, and other smart devices that are configured to monitor body parameters of a member.

In another embodiment, the method 600 includes modifying the generated one or more treatment processes recommendations based on processing of data relating to at least one of lab data including blood markers, allergy information, RNA, plasma, DNA, SNPs; activity data of the member; and processing data from one or more data from one or more biosensors configured in wearable smart device worn by the member. In an implementation, the treatment processes may be modified from time to time on receiving data from the lab tests or on receiving data from the bio sensors. The updated/modified recommendations of the treatment processes may be informed to the member as and when any change is required.

In an implementation, the method 600 includes generating one or more treatment processes recommendations under one of categories: no touch process, low touch process, and high touch process. In an implementation, the no touch process comprises one or more recommendations relating to at least one of sleep, mindfulness, physical activity, supplements, journaling including diet; the low touch process comprises one or more recommendations via an assigned healthcare professional; and the high touch process comprises one or more recommendations via at least one doctors.

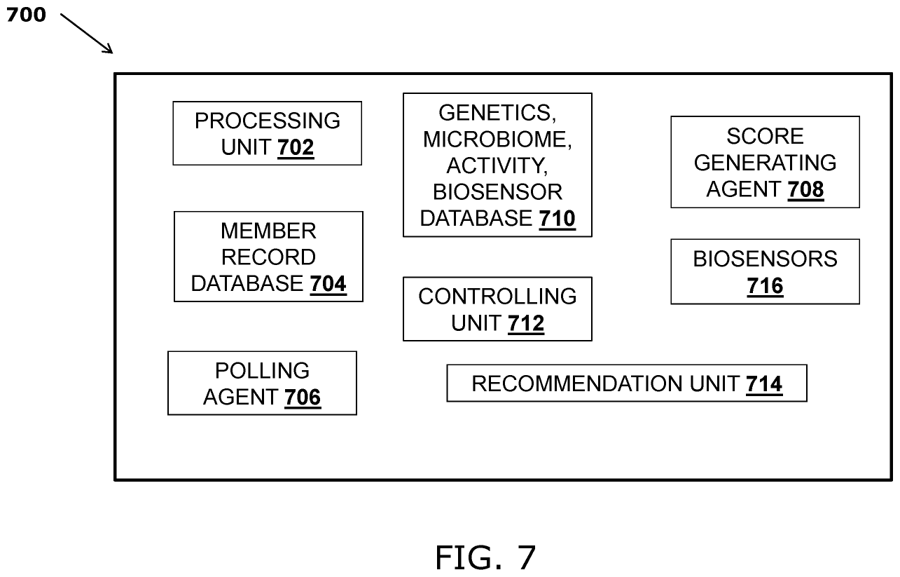

Referring to FIG. 7, a block diagram of an integrated healthcare monitoring system, in accordance with another embodiment of the present disclosure is provided. The integrated healthcare monitoring system comprises a processing unit 702 configured to accessing one or more information pertaining to a member from a member record database 704. The processing unit 702 is further configured to analyse the one or more information in respect of one or more attributes comprising: absences from work, performance reviews, relationship map, claims relating to behavioural issue, log-in hours, family history, age group, gender, performance improvement programs, prior work area, maternity period in case of females, social media behaviour, address, marital status, compensation, salary grade, skillset map, and current job profile. A polling agent 706 generates a first set of questionnaire to assess mental health of the member based on analysis said one or more information, and a score generating agent 708 generates a first score based on a response received from the member in respect of the first set of questionnaire, and a second score in respect of each of the one or more attributes based on analysis of said one or more information. The processing unit 702 is further configured to map the first score and second score respectively with a list of pre-defined scores in a pre-created matrix, said pre-created matrix including a mapping of one or more behavioural health information corresponding to a pre-assigned score, said one or more behavioural health information including at least one of severity level of mental health and one or more mental health underlying issues. The system 700 further includes a database 710 pertaining to genetics, microbiome tests which is accessed and processed by the processing unit 702 to identify one or more mental health underlying issues of the member. A controlling unit 712 ascertains if the one or more mental health underlying issues identified from the mapping correlate with the mental health underlying issues identified from processing of the data relating to the at least one of genetics, microbiome tests of the member. The system 700 further includes a recommendation unit 714 for recommending one or more treatment processes of a first type to be followed for restoration of the mental wellness of the member on basis the one or more mental health underlying issues confirmed on a positive ascertaining. The processing unit 704, polling agent 706, score generating unit 708, controlling unit 712, recommendation unit 714 may include one or more processors/microprocessors and microcontroller having processing and computation capabilities. The database 710 is also configured to store pre-created matrix, data relating to lab data including blood markers, activity data, data captured from biosensors which further assist in assessing the exact cause and the underlying mental health issue of the member.

In an implementation, based on data collected from various lab tests, microbiome tests, genetic data and other physical parameters data of cluster of members, the pre-created is formed based on the mental behaviour traits and a score is assigned in respect of each attributes affecting mental health.

In an exemplary implementation, system 700 fetches the employer data that provides initial indication of potential employees that have behavioral health issues. The system further requests a member to undergo one or more a wellness assessments to identify member archetypes. The system scores each question mapped to an archetype and then the overall scores across the archetypes are determined to derive the major/minor archetype based on employer data and wellness assessments. The system results from with behavioral health assessment to provide high likelihood of mental health issues with underlying issues based on archetypes. The behavioral assessment (e.g. PHQ-9, GAD-7, HAM-D etc. scientifically used by doctors today) provides a proven score if a person has depression/anxiety or other behavioral issues. The archetypes provide the underlying functional reasons for the mental health issue. The archetypes can further trigger genetics or microbiome tests based on the information provided to qualify the exact issue with either genetics or microbiome or both. Each of the archetypes questions can further be mapped to genetics, microbiome etc. and based on the scoring at that level between genetics, microbiome score, a genetic test or microbiome test can be triggered to confirm the issue with either genetics or microbiome. The output of the genetics/microbiome testing provides exact remediation process to help restore the mental wellness for genetics/microbiome issue and archetype recommendations. If genetics test or microbiome test are not available then the system is still configured to influence mental wellness based on member archetypes and recommendations associated with archetypes. In one implementation, the biosensors and activity data also enables the additional treatment methodology in addition to all the above.

The recommendations may include but not limited to, lifestyle recommendation of Nutrition, Exercise, Mindfulness, Sleep, Social style. Based on the analysis of the data from genetic, microbiome, biosensors, specific recommendation based on the archetype of the individual i.e. Digestion, Inflammation, Mitochondrial Dysfunction, HPA Axis, Hormone Imbalance, Stress may be provided.

In an implementation, a final health score is computed based on the assessments and results of analysis of the microbiome data, genetic data, lab data, biosensors data, wherein the health score assists in taking appropriate measures to improve the mental health and thus, the overall health score.

In an implementation, the system 700 analyses data from one or more of Full blood panel, microbiome (stool), DNA (spit), Medical history, Current symptoms. Current medicines, Current diagnoses, Current supplements, Family medical history, Lifestyle survey, Psych screen etc. to identify the mental health level and the underlying issues.

In an implementation, the system is configured to render and display results of lab tests, wellness assessments, employer data analysis, genetics test, microbiome tests and the appropriate recommendations to the member.

In an implementation, the wellness assessment questions include a Rule based question and answer flow that is focused on understanding the current lifestyle (Diet, Exercise, Sleep, Environmental, and Social) and Health style of the member. The lab data assists in confirming the underlying issues that trigger mental health condition. The genetics data assists to pinpoint the SNPs responsible and if they are active or not and the microbiome data assists to pinpoint the microbiome issues that could be elevating the issues. The data from the biosensors tracks member's activity data to pinpoint behaviours that needs to change to resolve the mental health problems of the member.

The present invention would be useful measuring productivity loss and improvement by trying to promote health and wellbeing for overall employee population and specific target to employees with behavioural health conditions such as Depression and Anxiety.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. An integrated health care monitoring system, the system comprising:
a user device communicatively coupled to at least one healthcare providers, wherein the user device is configured to access information, pertaining to a health of a member, from the at least one health providers, and wherein the information comprises electronic health records of the member pertaining to one or more of one or more diagnostics, therapeutics, key symptoms, signs, complications, or outcomes of a disease;
a member record database communicatively coupled to a member information portal and the user device, wherein the member record database is configured to:
receive, from the member information portal, information pertaining to demographic data comprising age, sex, geographical location, socio-economic classification, job classification, job performance data, absenteeism caused due to physical and mental health issues;
receive, from the user device, the information accessed from the at least one health providers, pertaining to the health of the member;
a member wellness artificial intelligence engine communicatively coupled to the member record database and one or more medical-based databases, wherein the member wellness artificial intelligence engine is configured to:
access, from the member record database, the information pertaining to the health of the member;
receive member-specific information from the one or more medical-based databases, wherein the member-specific information is relevant to the health of the member;
receive member genome data and member biome data from the at least one healthcare providers;
process the information received from the member record database, the received member genome data, the received member biome data, the information received from the medical-based databases to generate a propensity assessment for the member, wherein the propensity assessment comprises a probabilistic measure of future physical risks and mental health risks that the member is prone to;
a member results suite communicatively coupled to the member wellness artificial intelligence engine, the member results suite configured to:
process the propensity assessment to generate one or more recommendations for the member based on the physical risks and the mental risks that the member is prone to;
present the generated one or more recommendations on the user device under one of categories: no touch process, low touch process, and high touch process, wherein:
the no touch process comprises one or more recommendations relating to at least one of sleep, mindfulness, physical activity, supplements, journaling including diet;
the low touch process comprises one or more recommendations via an assigned healthcare professional;
the high touch process comprises one or more recommendations via at least one doctor.

2. The integrated healthcare monitoring system as claimed in claim 1, further comprising a member activity database configured to:
record actions of the member based on the one or more recommendations, wherein the actions of the member are recorded via one or more bio-sensors; and
update the member wellness artificial engine based on the recorded actions of the member in order to update the one or more recommendations generated for the member.

3. The integrated health care monitoring system as claimed in claim 1, further comprising a user interface pertaining to the user device, the user device configured to present health and wellness surveys and to receive information from the member, pertaining to a physical health, a mental health, and a social health of the member.

4. The integrated healthcare monitoring system as claimed in claim 1, further comprising third party platforms integrated with system, wherein the third party platforms comprise at least one of: Gyms, health monitoring applications, health monitoring smart watches, online shopping stores, pharmaceuticals and drug delivery applications, social networking applications.

5. The integrated healthcare monitoring system as claimed in claim 4, wherein the one or more recommendations comprise one or more of: targeted dietary changes and supplements, option to purchase targeted meals from one the integrated platforms, specify physical fitness activity regimen like to regulate specific condition, option to subscribe to one or more of integrated fitness platforms, improve engagement to create and align groups of members with common goals, interests or diet and/or physical fitness regimen, tele-medicine consultation with doctors on the targeted plan follow-ups, food and exercise log.

6. The integrated healthcare monitoring system as claimed in claim 1, wherein the member results suite is further configured to trigger an early intervention alarm via the user device, wherein the alarm is triggered when a health condition of the member is above a predefined critical condition.

7. The integrated user interface as claimed in claim 1, wherein the member wellness suite is further configured to:
share details of a progress in health of a first member with a second member via the user device;
schedule and synchronize one or more common activities for the first member and the second member via the user device;
assign wellness points to the first member and the second member, show avatars for the first member and the second member; and
compare a progress of the first member to the second member via the user device.

8. The integrated healthcare monitoring system as claimed in claim 1, wherein the member information portal is configured to analyse one or more attributes pertaining to a members to generate the information, and wherein the one or more attributes comprise: absences from work, performance reviews, relationship map, claims relating to behavioural issue, location of work, log-in hours, family history, age group, gender, performance improvement programs, prior work area, maternity period in case of females, social media behaviour, address, marital status, compensation, salary grade, skillset map, and current job profile.

9. The integrated healthcare monitoring system as claimed in claim 1, wherein the member wellness artificial engine is further configured to generate targeted recommendations based on the member genome data and the member biome data.

10. An integrated healthcare monitoring method, the method comprising:
accessing information, pertaining to a health of a member via a user device, from at least one health providers, and wherein the information comprises electronic health records of the member pertaining to one or more of one or more diagnostics, therapeutics, key symptoms, signs, complications, or outcomes of a disease;
receiving information pertaining to demographic data comprising age, sex, geographical location, socio-economic classification, job classification, job performance data, absenteeism caused due to physical and mental health issues from the member information portal;
receiving the information accessed from the at least one health providers, pertaining to the health of the member from the user device;
storing the received information in a member record database;
accessing the information pertaining to the health of the member from the member record database;
receiving member-specific information from one or more medical-based databases, wherein the member-specific information is relevant to the health of the member;
receiving member genome data and member biome data from the at least one healthcare providers;
processing the information received from the member record database, the received member genome data, the received member biome data, the information received from the medical-based databases to generate a propensity assessment for the member via a member wellness artificial intelligence engine, wherein the propensity assessment comprises a probabilistic measure of future physical risks and mental health risks that the member is prone to;
processing the propensity assessment to generate one or more recommendations for the member based on the physical risks and the mental risks that the member is prone to via the member results suite;
presenting the generated one or more recommendations on the user device under one of categories: no touch process, low touch process, and high touch process, wherein:
the no touch process comprises one or more recommendations relating to at least one of sleep, mindfulness, physical activity, supplements, journaling including diet;
the low touch process comprises one or more recommendations via an assigned healthcare professional;
the high touch process comprises one or more recommendations via at least one doctor.

11. The integrated healthcare monitoring method system as claimed in claim 10, further comprising recording actions of the member based on the one or more recommendations, wherein the actions of the member are recorded via one or more bio-sensors; and updating the member wellness artificial engine based on the recorded actions of the member in order to update the one or more recommendations generated for the member.

12. The integrated healthcare monitoring method system as claimed in claim 11, wherein the one or more recommendations comprise one or more of: targeted dietary changes and supplements, option to purchase targeted meals from one the integrated platforms, specify physical fitness activity regimen like to regulate specific condition, option to subscribe to one or more of integrated fitness platforms, improve engagement to create and align groups of members with common goals, interests or diet and/or physical fitness regimen, tele-medicine consultation with doctors on the targeted plan follow-ups, food and exercise log.

13. The integrated healthcare monitoring method system as claimed in claim 10, further comprising triggering to an early intervention alarm via the user device, wherein the alarm is triggered when a health condition of the member is above a predefined critical condition.

14. The integrated healthcare monitoring method system as claimed in claim 10, further comprising analysing one or more attributes pertaining to a members to generate the information, and wherein the one or more attributes comprise: absences from work, performance reviews, relationship map, claims relating to behavioural issue, location of work, log-in hours, family history, age group, gender, performance improvement programs, prior work area, maternity period in case of females, social media behaviour, address, marital status, compensation, salary grade, skillset map, and current job profile.

15. An integrated healthcare monitoring method, the method comprising:
accessing one or more information pertaining to a member from a member record database;
analysing said one or more information in respect of one or more attributes comprising: absences from work, performance reviews, relationship map, claims relating to behavioural issue, log-in hours, family history, age group, gender, performance improvement programs, prior work area, maternity period in case of females, social media behaviour, address, marital status, compensation, salary grade, skillset map, and current job profile;
generating a first set of questionnaire to assess mental health of the member based on analysis said one or more information;
generating a first score based on a response received from the member in respect of the first set of questionnaire, and a second score in respect of each of the one or more attributes based on analysis of said one or more information;
mapping the first score and second score respectively with a list of pre-defined scores in a pre-created matrix, said pre-created matrix including a mapping of one or more behavioural health information corresponding to a pre-assigned score, said one or more behavioural health information including at least one of severity level of mental health and one or more mental health underlying issues;
accessing and processing data relating to at least one of genetics, microbiome tests of the member to identify one or more mental health underlying issues of the member;
ascertaining if the one or more mental health underlying issues identified from said mapping correlate with the mental health underlying issues identified from processing of the data relating to the at least one of genetics, microbiome tests of the member; and
recommending one or more treatment processes of a first type to be followed for restoration of the mental wellness of the member on basis the one or more mental health underlying issues confirmed on a positive ascertaining.

16. The integrated healthcare monitoring method as claimed in claim 15 further comprising accessing and processing data relating to at least one of lab data including blood markers, allergy information, RNA, plasma, DNA, SNPs; and activity data of the member for ascertaining if the one or more mental health underlying issues identified from said mapping correlate with the mental health underlying issues identified from processing of the data relating to the at least one of genetics, microbiome tests of the member.

17. The integrated healthcare monitoring method as claimed in claim 15, further comprising accessing and processing data from one or more data from one or more biosensors configured in wearable smart device worn by the member, wherein the data from the biosensors includes data relating to breathe and oxygen levels, heart beat level, glucose, stress levels, mood, anxiety levels.

18. The integrated healthcare monitoring method as claimed in claim 15, further comprising generating one or more treatment processes recommendations under one of categories: no touch process, low touch process, and high touch process, wherein:
the no touch process comprises one or more recommendations relating to at least one of sleep, mindfulness, physical activity, supplements, journaling including diet;
the low touch process comprises one or more recommendations via an assigned healthcare professional; and
the high touch process comprises one or more recommendations via at least one doctor.

19. The integrated healthcare monitoring method as claimed in claim 18, further comprising modifying the generated one or more treatment processes recommendations based on processing of data relating to at least one of lab data including blood markers, allergy information, RNA, plasma, DNA, SNPs; activity data of the member; and processing data from one or more data from one or more biosensors configured in wearable smart device worn by the member.

20. The integrated healthcare monitoring method as claimed in claim 18, wherein the one or more treatment processes recommendations are based on archetype of the member, said archetype including Digestion, Inflammation, Mitochondrial Dysfunction, HPA Axis, Hormone Imbalance, Stress.
